(19) United States
(12) Patent Application Publication
(10) Pub. No.: US 2025/0224096 A1
(43) Pub. Date: Jul. 10, 2025

(54) Illumination System

(71) Applicant: SCHOTT AG, Mainz (DE)

(72) Inventors: Bernd Schultheis, Mainz (DE); Jens Vietor, Mainz (DE); Albrecht Seidl, Mainz (DE); Philipp Muders, Mainz (DE)

(73) Assignee: SCHOTT AG, Mainz (DE)

(21) Appl. No.: 19/004,803

(22) Filed: Dec. 30, 2024

(30) Foreign Application Priority Data
Jan. 4, 2024 (DE) 10 2024 100 198.4

(51) Int. Cl. F21V 9/32; F21L 4/00; F21V 8/00

(52) U.S. Cl. CPC F21V 9/32; F21L 4/005; G02B 6/0008

(57) ABSTRACT

An illumination system is provided, having a handpiece with an illumination unit and a semiconductor lighting element which provides a radiation of essentially blue light in the wavelength between 400 nm and 500 nm, and a light guide unit connectable to the handpiece. The light guide unit includes a fiber-optic light guide rod, having a proximal end face and a distal end face, and a sleeve. The fiber-optic light guide rod is fixed in the sleeve by a first adhesive in a portion of the proximal end face. The light guide unit has a converter, for converting the radiation of a semiconductor lighting element in the operating state into a distal radiation of the fiber-optic light guide rod at its distal end face. The distal radiation in the operating state has essentially white and/or color-neutral light, or colored light, a wavelength in the visible spectral range.

### CROSS REFERENCE TO RELATED APPLICATIONS

This application claims the benefit of priority from German Patent Application No. 10 2024 100 198.4, filed Jan. 4, 2024, the disclosure of which is incorporated herein by reference.

### FIELD OF THE INVENTION

The invention relates to an illumination system comprising at least one handpiece having an illumination unit and at least one semiconductor lighting element, which in the operating state has a radiation of essentially blue light in the wavelength range between essentially 400 nm and 500 nm, and a light guide unit which is releasably connected and/or connectable to the handpiece and which comprises a fiber-optic light guide rod and a sleeve, the fiber-optic light guide rod being fixed into the sleeve by means of a first adhesive in the sleeve, or adhesively bonded thereto or adhesively bonded therein, and which in the operating state has a radiation of essentially white and/or color-neutral light, or colored light, of at least one wavelength or of at least one wavelength range in the visible spectral range, at the distal end of the light guide rod, that is to say the end facing away from the illumination unit.

### BACKGROUND OF THE INVENTION

Such illumination systems are used in particular to cure tooth fillings in the field of dentistry. In this case, the blue light output from the handpiece is guided through for example a glass-fiber rod to the tooth and cures the tooth filling material, which conventionally consists of a mixture of photocuring polymers and glass or glass-ceramic powder, within a few seconds.

Such apparatuses are known, for example, from DE 101 04 579 B4 or EP 3579786 B1.

DE 101 04 579 B4 describes a light curing apparatus which comprises a semiconductor radiation source, which is mounted in the light curing apparatus and radiates radiation at least partially in the visible spectral range and can be switched on in order to cure a compound lying in the beam path. The radiation has an illumination intensity of at least 200, in particular at least 300 mW/cm², and the semiconductor radiation source is in metallic and/or ceramic thermally conductive connection with a base body. Typically, a peak wavelength of 440 nm and 470 nm is used.

EP 3579786 B1 discloses a dental light irradiation device comprising a first light source for emitting blue light, typically in the range of from 430 to 480 nm, a camera and at least a first detector, wherein the device is operable in a camera mode (C) in which the camera is activated and in a non-camera mode (N) in which the camera is deactivated, wherein in the non-camera mode (N) a user input on the first detector activates the camera mode (C), wherein detection of a “freeze image” instruction of the user input via the first detector in the camera mode (C) triggers a freeze of an image captured by the camera and detection of a “camera off” instruction of the user input via the first detector causes the device to switch into the non-camera mode (N). In addition, in this device a white-emitting 2nd light source may be switched on in the camera mode (C) instead of the blue-emitting light source. This is a complex and elaborately configured apparatus, which besides the curing of tooth fillings also enables visual assessment, including image documentation.

For the inspection of teeth, for example, US2018256033 A1 discloses an approach in which a tooth crack observation tip is provided and the tip comprises a tip body which is inserted into a coupling tube of a light curing device from which a light guide tip has been separated; a crack observation guide tube which is formed to extend from the tip body by a predetermined length and is inserted into the mouth. The unit furthermore comprises a light conversion filter, which is coupled to a rear end of the tip body and converts blue light into white light, the light conversion filter being formed by coating a red color and a green color on both sides of the tip body. A disadvantage with this approach is that the light conversion filter must be placed individually between the tip and the light curing device, which is comparatively elaborate. A high proportion of light is also lost by absorption owing to the two color filters.

### OBJECT OF THE INVENTION

It is therefore an object of the invention to provide an illumination system which for practical operation, that is to say for example in the context of dental use, provides white or colored light mechanically robustly and with high efficiency while enabling applications such as “curing” of fillings, “inspection” or “assessment” of teeth or gums, or “examination” for example of tissue or modification thereof, particularly in the oral cavity, without additional apparatuses, in particular further or separate illumination instruments, while enabling or ensuring simple and reliable handling, a compact design and a simple change between the various application cases. It is furthermore desirable, as far as possible with existing equipment for the curing of tooth fillings, also to carry out the applications additionally mentioned above with colored and/or white light without investing in additional, usually expensive, special apparatuses.

### SUMMARY OF THE INVENTION

The object of the invention is already achieved by the subject matter of the independent claims. The dependent claims relate to advantageous configurations and developments.

The invention provides an illumination system which comprises a handpiece having an illumination unit and at least one semiconductor lighting element, wherein the at least one semiconductor lighting element has in the operating state a radiation of essentially blue light in the wavelength range between 400 nm and 500 nm, and further a light guide unit which is releasably connected and/or connectable to the handpiece. The light guide unit comprises a fiber-optic light guide rod, having a proximal end face and a distal end face, and a sleeve. The fiber-optic light guide rod is fixed in the sleeve by means of a first adhesive in a portion starting from the proximal end face, that is to say on its lateral faces. The light guide unit comprises at least one converter, the converter converting the radiation, that is to say for example blue light, of the at least one semiconductor lighting element in the operating state into a distal radiation of the fiber-optic light guide rod at its distal end face, so that this distal radiation in the operating state has essentially white and/or color-neutral light, or colored light, of at least one wavelength or of at least one wavelength range in the visible spectral range.

In other words, the light guide unit has at least one converter which, in the operating state, converts the radiation of the semiconductor lighting element in the direction of the proximal end of the fiber-optic light guide rod into a distal radiation of the fiber-optic light guide rod, in such a way that this distal radiation essentially corresponds to white light or colored light of at least one wavelength or wavelength range of the visible spectrum of electromagnetic radiation, for example in the red, green, blue or yellow spectral range, or a mixture of at least two colors therefrom. Such converters are also referred to as wavelength converters, and thus make it possible for example to convert the light of an exciting first wavelength (primary light) into light in particular of a second wavelength emitted by the converter (secondary light), this wavelength being different from that of the primary light. Besides the conversion of primary light into secondary light in the visible spectrum of electromagnetic radiation, it is also possible by means of corresponding converters to convert it into secondary light, or secondary radiation, for example of at least one wavelength or wavelength range of the infrared (IR) or ultraviolet (UV) spectrum of electromagnetic radiation.

Depending on the application, a plurality of materials converting into different wavelengths may also be used, and light may thus be provided in a plurality of ranges of the visible light spectrum so that differently colored light may in particular also be obtained in their mixture.

With corresponding light mixing by a converter material designed suitably, predominantly in respect of porosity, doping and thickness, white light may thus also be generated. This is possible for example with blue light sources of a first wavelength or wavelength range in combination with yellow light of a second wavelength or wavelength range of a converter, or converter material, by corresponding mixing of the blue light, which excites the converter and is partially converted into conversion light, and of the converted yellow light thus obtained. A system which is mechanically robust for practical operation may thus be produced, which on the one hand converts the blue light into white or colored light with high efficiency and on the other hand enables a simple change between the applications of “curing”, in particular with blue light, and “inspection”, “assessment” or “examination”, for example with white or colored light, by the normal light guide unit used for the curing, without a converter, being replaced with a light guide unit having a converter.

Besides blue light in the visible spectral range, as described in the introduction, such conversion is in principle also possible when using or employing a corresponding converter material with UV light having a wavelength <400 nm.

If the converter is located at the proximal end face of the fiber-optic light guide rod and overlaps the latter at least partially, as is the case in a particularly preferred embodiment, and is therefore located in the immediate vicinity of the illumination unit of the handpiece when the light guide unit is inserted into the handpiece in order to operate the dental illumination unit, then the emitted blue light which therefore illuminates or shines through the converter at the proximal end of the light guide rod can be converted with high efficiency into white light or colored light and be provided at its distal end. Also, the converter may be installed so that it is protected by the sleeve in this case, and does not come in contact inter alia with tissue of the patient during use.

In one advantageous embodiment of the illumination system, as an alternative or in addition, the converter is accordingly assigned to the proximal end face of the fiber-optic light guide rod and at least partially overlaps the proximal end face. When the light guide unit and the handpiece are connected, the converter is therefore arranged facing towards the illumination unit.

In a preferred embodiment, the converter comprises or consists of a ceramic material, the radiation of the illumination unit in transmission being influenceable by the converter material in the operating state. For an essentially white or color-neutral distal radiation at the distal end face of the fiber-optic light guide rod, the converter comprises or consists of Ce:YAG, for colored radiation with a centroid in the red or red-yellow spectral range of visible light, the converter comprises or consists of Ce:YAG with additional gallium (Ga) doping as co-doping, or for colored radiation with a centroid in the green spectral range of visible light, the converter comprises or consists of Ce:LuAG. By additional doping of Ce:LuAG with gadolinium (Gd), the centroid of its colored radiation may be shifted even further into the green range. Ce:YAG stands for cerium-doped yttrium aluminium garnet, and Ce:LuAG stands for cerium-doped lutetium aluminium garnet. As an alternative or in addition, the converter may also consist of or comprise nitride or oxynitride ceramic phosphors, usually doped with europium, for example of the type Eu:SiN, Eu:SiON and/or Eu:SiAlON, which in particular emit secondary light in the green and red wavelength range of the visible spectrum and may likewise be co-doped with additional elements, for example calcium and/or barium, in order to modify the emission wavelength. By using corresponding conversion materials, it is also possible to achieve conversion of primary light into secondary light, or secondary radiation, for example into light or radiation in the infrared (IR) or ultraviolet (UV) range of the spectrum of electromagnetic waves.

These ceramic materials as converters have, in particular, a high thermal stability, particularly compared with plastic-based converter materials.

As a further alternative, the converter, or the converter material, may also be formed as a material composite of a matrix with particles of at least one phosphor incorporated therein. The converter may therefore also consist of a ceramic matrix, glass matrix or a polymer matrix, or plastic matrix, for example a silicone compound or epoxy resin, in which one or more so-called phosphors, that is to say fluorescent conversion substances, are incorporated. When excited by light of one wavelength or wavelength range (primary light), such phosphors can emit light of a different wavelength or wavelength range (secondary light). The terms phosphor-in-glass, phosphor-in-ceramic or phosphor-in-silicone are also used in this context. These differ significantly from the ceramic converter materials, as is revealed in particular by the material properties and is represented in the following Table 1 with the aid of selected properties:

TABLE 1

| | Ceramic | Phosphor- | Phosphor- |
|---|---|---|---|
| Property | converter | in-glass | in-silicone |
| Thermal conductivity | 6 to 10 W/mK | 0.8 to 1.2 W/mK | 0.2 to 1 W/mK |
| Thermal stability | >1000° C. | up to about | up to about |
| | | 700° C. | 160° C. |
| | (up to 250° C. with organic binding) | | |
| Robustness | high | high | low |

A high thermal conductivity is advantageous in order to avoid overheating of the converters or at least to minimize their thermal stress, which is relevant in particular for high light intensities, for example when the converters are excited, or illuminated, with high-power lasers. The resulting heat may then be dissipated with high efficiency, for example to cooling faces corresponding to the converter. Concomitantly, a high thermal stability is also of interest, and is advantageous in particular for ceramic converters as well as for a phosphor-doped glass matrix. Overheating of the converter material may lead to undesired color location shifts of the emitted light, and above all also to a reduced luminous efficiency due to so-called thermal quenching of the converter material, or of a phosphor.

The robustness relates here both to the mechanical stability, or strength, and in particular to chemical stability, which is significant above all if, during the disinfection, sterilization or reconditioning that are conventional in medical technology or medical applications, a converter is exposed to the processes and/or chemicals necessary therefor, or comes into contact therewith.

The converter may furthermore comprise or consist of a combination of at least two converter materials. Such a combination is important particularly in the case of the composite materials described above as converter material, such as phosphors in glass or in silicone.

In a further advantageous embodiment of the illumination system, the converter accordingly comprises or consists of at least one of the following materials: a ceramic converter material of the type Ce:YAG, a ceramic converter material of the type Ce:YAG with additional Ga doping, a ceramic converter material of the type Ce:LuAG, a ceramic converter material of the type Ce:LuAG with additional Gd doping, a ceramic converter material of the type Eu:SiN, Eu:SiON and/or Eu:SiAlON, a converter material as a material composite of a glass matrix with particles of at least one phosphor incorporated therein, a converter material as a material composite of a ceramic matrix with particles of at least one phosphor incorporated therein and/or a converter material as a material composite of a polymer matrix with particles of at least one phosphor incorporated therein. The converter may also comprise or consist of a combination of at least two of the aforementioned converter materials.

Such converters are generally somewhat translucent to opaque, and only in small thicknesses sufficiently transparent to convert primary light in transmission to secondary light, or provide the latter for example for illumination purposes. The nature of the converter, for example the material of the converter, or its conversion properties, and the configuration, for example its thickness, make it possible to adjust or control the color or the color impression of the light provided overall. The latter may also comprise a mixture of primary and secondary light. Such converters may be used both in transmission, i.e. transmissively, or in remission, i.e. remissively. Here, transmissive means that the primary light shines through the converter from a first surface to a second surface while being converted at least partially into secondary light, which is then provided in particular at or after the second surface. In remission, a surface of the converter is illuminated with primary light, which penetrates it at least partially while being converted at least partially into secondary light and is provided by backscattering in particular at or after the illuminated surface. In both cases, depending on the conversion ratio of primary light into secondary light, a mixture of the two may be provided, or else only secondary light may be provided in the case of complete conversion.

For transmissive use of the converters, which are often somewhat translucent, thicknesses of 100 μm or less, typically 80 μm are particularly advantageous. By corresponding finishing, for example grinding, lapping or polishing, thicknesses of from 50 μm down to 30 μm may also be prepared in order to render them more transparent. Depending on the thickness of the converter and/or the nature of the phosphor which it comprises, the primary light entering the converter is converted at least partially into secondary light so that, after transmission of primary light and secondary light through the converter, there is mixed light consisting of the two, or else only secondary light in the case of complete conversion of the primary light. Such conversion elements may for example be produced from a wafer by mechanical, abrasive or machining methods such as sawing or boring processes, particularly advantageous by laser processing in arbitrary contours (for example square, rectangular, multi-angled, polygonal, round or non-round). For the application according to the invention, circular discs having a diameter of from 4 mm to 12 mm are preferred, this corresponding substantially to the diameter of the fiber-optic light rod, which is generally likewise produced in a round form.

The converter may also be composed of, or constituted by, a plurality of converter elements, also consisting of different converter materials, optionally each with different thicknesses of the respective converter elements. Thus, on the one hand, a plurality of converter elements may be arranged in a stack of thin platelets and/or on the other hand may also be constituted by segments, that is to say for example a round converter may be composed of a plurality of circle segments, which may also each consist of different converter materials. As described above, the converter overlaps the proximal end face of the light guide rod at least partially, although it may also cover or overlap it fully. Only partial coverage of the proximal end face of the light guide rod by the converter may be desired or preferred when a particular, optionally predetermined mixture of primary and secondary light is intended to be achieved. In such a case, but also in general, mixing or homogenizing of the light coupled proximally into it takes place at least partially in the light guide rod towards its distal end.

Accordingly, it is particularly advantageous for the converter of the illumination system to be formed as an alternative or in addition as one or more thin disc-shaped converter elements consisting of at least one converter material, which at least partially cover the proximal end face of the fiber-optic light guide rod and each of which has a thickness of from 30 μm to 200 μm, preferentially from 50 μm to 150 μm, particularly preferentially from 80 μm to 100 μm.

Additional measures, for example varying the cross-sectional area of the light guide rod by tapers or widenings may also influence the distal radiation characteristic, for example its radiation angle, or enable adjustment thereof, and may thus also promote the proximal coupling into the light guide rod.

In an advantageous configuration of the illumination system, as an alternative or in addition, the one or more converter elements of the converter are adhesively bonded by means of a second adhesive onto the proximal end face of the fiber-optic light guide rod and/or to one another, in particular without any bubbles, and this second adhesive is substantially optically clear, transparent and permanently elastic in the cured, or processed or crosslinked, state.

In the context of the invention, optically clear, transparent means here that the second adhesive substantially does not have any color of its own, does not have a scattering effect in the wavelength range of the light passing through the adhesive and causes no attenuation, or at least no substantial attenuation, of the light passing therethrough, that is to say it has for example at least a transmission of 90% at a thickness of 1 mm for these wavelengths, or in this wavelength range. Bubble-free adhesive bonding is important particularly when the illumination unit has at least one light source which, because of its power, locally or extensively radiates heat, or introduces it into the converter at the proximal end, in such a way that any bubbles present may expand excessively and this may lead to damage, delamination even to the extent of destruction of the adhesive bond.

Also, this second adhesive may be matched in respect of its refractive index to that of the fiber-optic light guide rod, or may differ therefrom only very little, for example Δn≤0.1. This reduces reflection losses at the respectively associated interfaces.

In respect of the processing, it may also be advantageous for the second adhesive to have a low viscosity, or self-levelling flow properties, during application and to be processable, or curable, by crosslinking with UV light and/or heat. This second adhesive therefore optionally differs from the first adhesive with which the fiber-optic light guide rod is adhesively bonded into the sleeve of the light guide unit. The first adhesive may satisfy such optical requirements, although it does not have to. Conversely, it may be advantageous in respect of the first adhesive if it is also provided in a translucent or opaque and/or colored, for example black-colored, form in order to avoid or at least reduce any scattered light from or around the sleeve.

During conversion into, or provision of, colored and/or essentially white light as distal radiation, it is advantageous that the color location when colored distal radiation is desired, or the color temperature when white and/or color-neutral distal radiation is desired, can be adjusted, or is adjustable, according to requirements and/or purposely by the composition of the converter, by the thickness of the converter or of the converter elements and the porosity of the converter material. For example, the color location of the distal radiation, particularly when ceramic converters based on yellow-emitting Ce:YAG are used with a blue excitation wavelength, is shifted towards cool white light, i.e. white light with a high color temperature (>5000 K as cool white), when the converter is made thinner since the blue light component passing through the converter is larger in this case. With a thicker converter, more neutral white light or even warm white light, i.e. white light with a low color temperature (typically in the range of from 4000 K to 5000 K as neutral white; typically <3000 K as warm white), may be obtained from the same converter material here.

Further adjustment possibilities are offered by material-related adaptations of the converter material, for example by increasing or lowering the doping levels, that is to say for example in the case of a Ce:YAG phosphor, or ceramic Ce:YAG converter material, increasing or lowering its content of Ce and/or optionally further components or co-dopings. This, as described above, for example as Ga co-doping. The conversion properties may therefore be adjusted by modifying the composition of the converter material. The conversion into, or provision of, colored and/or essentially white light as distal radiation may likewise be influenced by the porosity of the converter, or of the converter material.

In the case of composite materials as converters, the volume fraction of phosphors introduced as particles or powder into the associated matrix also plays a role in respect of the composition of the converter material. These are often provided in particle sizes of less than one μm, a few μm to a few tens of μm. The larger the volume fraction of phosphors is in this respect, the more, or more strongly, primary light is converted into secondary light. The particles of the phosphor, or else the interfaces between the particles and the matrix, moreover act as scattering elements in these composite materials, in particular for the secondary light but also possibly unconverted primary light.

In the aforementioned converters, or their materials, that is to say not only in composite materials but also the ceramic converters, their scattering may likewise be influenced by their porosity. Thus, by means of the porosity, both the path of the exciting primary light and the path of the emitted secondary light may be modified. Therefore, the radiation of the converter, that is to say the color location when distal radiation of colored light overall is desired, or the color temperature when essentially white radiation is desired, may thus be varied or adjusted by means of the porosity of the converter materials, or of the converter elements, the interface between pores and material surrounding them in particular also having a scattering effect. Thus, for example, if a ceramic converter, or converter element, which comprises or consists of Ce-doped YAG with a particular composition is illuminated or shone through with blue excitation light from one side, it will emit yellow light which, with a suitably adjusted thickness and particular porosity of the converter, will for example give neutral white light on the side shone through. If the porosity is then increased, the color temperature is shifted in the direction of warmer white, and in the converse case of a reduction of the porosity, towards cooler white. With a constant porosity of this converter, the color temperature of white light may likewise be varied by varying the thickness, i.e. with a reduced thickness in the direction of cooler white or with an increased thickness towards warmer white.

Accordingly, in advantageous embodiments of the illumination system, the color location in the case of colored distal radiation or the color temperature in the case of white or color-neutral distal radiation is adjustable by the composition of the converter and/or of the one or more converter elements, by the thickness of the converter and/or of the one or more converter elements, and/or by the porosity of the converter and/or of the one or more converter elements, i.e. they can be adapted in this way.

Thus, particularly in the white range, depending on the configuration of the converter, both cool white light (color temperature approximately >5000 K), neutral white light (color temperature of approximately between 4000 K and 5000 K), or warm white light (color temperature of approximately <3000 K), or else color-neutral light may be created. Color-neutral light in this case specifically means light which in the L-a-b color space is essentially located along the L axis, or for a given L value lies around the L axis with a small color deviation Delta E of in particular <2, preferably <1.

In a further advantageous embodiment of the illumination system, as an alternative or in addition a cover is applied on the converter, or on the outer-lying converter element, in such a way that the converter, or the outer-lying converter element, is at least fully covered or overlapped, this cover comprising or consisting of a glass disc, a plastic disc, a sapphire disc or a quartz glass disc and having a thickness in the range of from 30 μm to 500 μm, preferably from 50 μm to 200 μm, and this cover likewise being connected to the converter with the second adhesive. If the converter element is constructed from a plurality of converter elements, optionally of different converter materials, the outer-lying converter element is the converter element which is the last of at least two to have been arranged, or applied, onto the proximal end of the light guide rod or with the greatest distance therefrom.

This disc-shaped cover, or cover disc, serves to protect the converter, particularly when it or at least one of the converter elements is adjusted to be comparatively porous. Chemical and mechanical protection of the converter may therefore be achieved particularly in the conditioning methods that are usual in the medical field, for example autoclaving (typically up to 140° C., >3 bar steam and as much as a few to a few tens of minutes of exposure time per cycle, number of cycles >>100). The materials of the cover correspondingly need to be rendered chemically resistant, as well as optically in such a way that substantially unimpeded incidence of the light of the illumination unit is possible in the operating state, without scattering by the cover. The cover, or its material, is thus to be rendered clear, transparent, i.e. as already described above in respect of the second adhesive, substantially non-scattering and colorless or without any color of its own, particularly in the relevant wavelength range. The cover thus, for example, has a transmission of more than 80% at a thickness of 1 mm, at least in this wavelength range. For example, thin platelets of borosilicate glass have proven suitable as a preferred cover. The cover accordingly closes the proximal end of the light guide rod, on which the converter is arranged, optionally in the surrounding sleeve, and in the state fitted into the handpiece the cover is therefore arranged facing towards the illumination unit located in the handpiece with its light source. Overall, the attachment of a cover to the converter furthermore increases the robustness and practicability of the system which were described in the introduction.

Alternatively or additionally, it is advantageous for the cover disc that its coefficient of thermal expansion is matched to that of the converter respectively the converter element, at least the outer-lying one, wherein the difference between the coefficients of thermal expansion is less than 6.0 ppm/K, preferably less than 4.5 ppm/K, most preferably less than 3.5 ppm/K. Ideally, the thermal expansion coefficients of the cover disc and the converter material differ by no more than 1 ppm/K and are preferably no greater than 10 ppm/K. Typically, ceramic converters of the Ce:YAG type, for example, have a coefficient of thermal expansion of around 6.5 ppm/K, while those of nitride phosphors, such as those of an Eu:SiN, Eu:SiON and/or Eu:SiAlON type, have a coefficient of thermal expansion of around 3 ppm/K. Glasses suitable for the cover disc comprise or consist of, for example, borosilicate glasses such as D263® with a coefficient of thermal expansion around 7.2 ppm/K, Borofloat33®, Mempax® or AF32® with a thermal expansion coefficient of 3.2 to 3.3 ppm/K from SCHOTT AG. Also suitable are glasses with a coefficient of thermal expansion of around 8 ppm/k to around 9.5 ppm/K, such as soda lime glasses or B270®, Xensation®, AS87® from the applicant.

Any remaining differences in the coefficients of thermal expansion can be compensated for by the second adhesive. Such an adjustment of the coefficients of thermal expansion favours the robustness and lifespan of the system and prevents possible damage due to thermomechanical stress, such as that which can occur during the above-mentioned conditioning methods (i.a. for disinfection-, sterilisation-purposes), or at least delays such possible damage.

In order to avoid reflection losses, in a further advantageous embodiment of the illumination system, as an alternative or in addition, the converter and/or the cover has a reflection-reducing coating at least on the side facing towards the illumination unit. Such layers are for example so-called N/4 layers (for example approximately 115 to 120 nm thick at a light wavelength of 450 nm) or consist, for example, of a sequence of a plurality of layers of SiO₂ in alternation with TaO₂.

In a further advantageous configuration of the illumination system, as an alternative or in addition, a color filter may be arranged between the converter and the proximal end face of the fiber-optic light guide rod. Such color filters may be provided for fine adaptation, or requirement-based adjustment, of the distal radiation of the fiber-optic light guide rod, for example of color values and/or color locations in particular for colored distal radiation, or color temperature in particular for essentially white or color-neutral distal radiation. This color filter may, for example, be provided as filter glass platelets between the converter and the proximal end of the fiber-optic light guide rod and/or as a layer system on the fiber-optic light guide rod or on the converter, on the side of the latter facing towards the fiber-optic light guide rod. Thus, a particular wavelength range may be filtered out from the converted light, or the light provided after the converter in the direction of the proximal end face of the light guide rod. Naturally, such a color filter may in principle also be provided on the distal end face of the fiber-optic light guide rod, although this would be less preferred with regard to the robustness of the system. Preferentially, such a color filter is likewise fixed at the correspondingly intended position by means of the second adhesive.

In a further advantageous embodiment of the illumination system, as an alternative or in addition, the sleeve has a widening of the inner diameter in the region of the converter and/or of the cover, so that an adhesive reservoir is formed or formable. This facilitates, simplifies, or improves the assembly and makes it possible to avoid an excess of adhesive which may otherwise remain on the converter, or on the cover, and would interfere with the optically active faces. Thus, volume tolerances in the dosing of the second adhesive may therefore also be compensated for so that application of adhesive onto the optically relevant faces of the converter or of the cover may be avoided.

Preferred uses of the illumination system are, in particular, those for the visual assessment of tooth fillings and/or the gums, for the visualization of plaque or tissue modifications, particularly in the oral cavity, and for the visual assessment of adhesive processes and/or adhesive curing in medical or industrial applications. The visual assessment of tooth fillings and/or gum assessment essentially presupposes white light, with colored light also being employed in order to visualize plaque or tissue modifications. In general, visual tissue examinations in the mouth and throat may thus also be carried out with such an illumination system, for example on the gums, on the tongue and on the surfaces of the teeth, under different types of illumination. This is also made possible by the easy changing of the light guide unit, which may be inserted into the handpiece with the illumination unit, or replaced, depending on the necessary or requested type of illumination with white or colored light, the light guide units respectively being equipped with corresponding converters, although they may optionally also be provided without converters.

Further medical or industrial adhesive bonding processes or examinations may also be carried out or operated with an illumination system according to the invention, for example in which adhesive curing initially takes place with blue light and then a visual assessment is intended or needs to be carried out with white or colored light after complete adhesive curing has been carried out. Examples, albeit without limitation thereto, from the medical field are vascular surgical adhesive bonding processes, for example of skin or bone. In the industrial sector, these adhesive bonding processes may be in the field of electronics, in which, for example, contact adhesive bonds need to be subjected to a visual inspection after adhesive bonding has been completed.

### BRIEF DESCRIPTION OF THE DRAWINGS

The invention will be described in more detail below with the aid of figures, in which

FIG. 1 shows a schematic structure of an illumination system consisting of a handpiece and a light guide unit,

FIG. 2 shows a detail enlargement in the region of the proximal end of the light guide unit, and

FIG. 3 shows a CIE color space diagram of the color location modification by the converter.

### DETAILED DESCRIPTION OF THE INVENTION

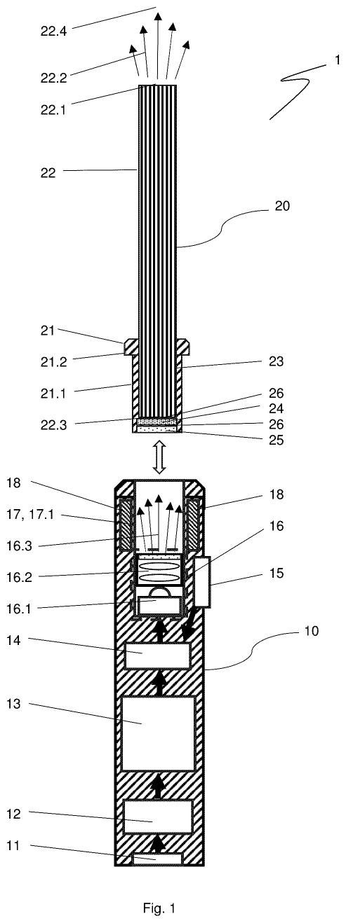

FIG. 1 schematically shows a sectional representation of the structure of an illumination system 1 according to the invention.

The illumination system 1 consists of a handpiece 10 and a light guide unit 20 releasably connected or connectable, or pluggable, thereto, in which case the handpiece 10 conventionally has a charging interface 11 for charging a corresponding energy storage unit 13 and the charging may be monitored, or the state of charge may be visually displayed, with a charge monitoring unit 12. The energy storage unit 13 is usually formed by rechargeable batteries, that is to say accumulators or secondary batteries. Alternatively, conventional batteries, that is to say primary batteries, may also be envisaged although they are less suitable for practical operation. A cabled energy supply may also be envisaged. An illumination unit 16 can be driven via an illumination control unit 14, which has at least one operating element 15. The start time, the light intensity and the duration of the illumination may in this case conventionally be adjusted, or specified by the user.

The illumination unit 16 conventionally consists of one or more semiconductor lighting elements 16.1 in the form of one or more LEDs or laser diodes, or combinations of the two. An optical element 16.2 is often also provided, which concentrates or collimates the radiated light of the semiconductor lighting element 16.1 so that ideally most, or the predominant part, of the light that is radiated by the one or more semiconductor lighting elements 16.1 in the operating state can be coupled into the light guide unit 20 and the intensity of the light provided, particularly at the distal end of the light guide unit, is therefore increased and for example the treatment duration, for example for the curing of light-curing tooth fillings, may thereby be shortened. The optical element 16.2 conventionally consists of a lens or a lens system, and may additionally comprise a planar cover disc as protection.

Since this handpiece 10 is used to cure tooth fillings that are cured by light or crosslinked by light in the blue or near UV range, in the operating state the radiation 16.3 of the illumination unit 16 is situated in the blue spectral range of visible light between 400 nm and 500 nm. Depending on the curing mechanisms of the tooth filling materials used, or their crosslinkable resin systems, in particular the wavelengths 405 nm and 450 nm are of interest in order to achieve rapid and complete curing, or crosslinking, of the tooth filling. Other wavelengths are however also common, as described in the introduction. Often therefore, semiconductor lighting elements 16.1 radiating different colors are also used in an illumination unit 16 in order to achieve an optimal short curing time overall but also complete curing.

The handpiece 10 also has a reception portion 17 for receiving the light guide unit 20, a geometrically accurate fit 17.1 being provided, which corresponds with the fit 21.1 of a sleeve 21 of the light guide unit 20. What is crucial for the design of the fits 17.1 and 21.1 is in this case to ensure firm, secure seating of these components in or on one another despite the releasability of the two modules. Assisting this or as an alternative (although not represented in FIG. 1), seals in the form of for example O-rings may be provided, which serve for further fixing as well as for sealing. In modern handpieces 10, there may also be magnets 18 in the region of the reception portion 17, which correspond in terms of force with magnetizable inlays (for example consisting of magnetizable stainless steel) in the sleeve 21 of the light guide unit 20 and thus likewise ensure firm holding of the light guide unit 20 in the handpiece 10 during operation.

The essential item of the light guide unit 20 is formed by a fiber-optic light guide rod 22, which is conventionally adhesively bonded with an accurate fit into the sleeve 21 by means of a first adhesive 23. To this end, translucent or opaque, in particular blackly opaque adhesives based on epoxide or silicone are conventionally used. Besides a shaft, which corresponds with the reception portion 17 of the handpiece 10, the sleeve 21, which conventionally consists of stainless steel or a thermally stable plastic (for example polyphenylsulfone PPSU or polyphenylene sulfide PPS, these optionally also with glass fiber reinforcement), also has a stop 21.2 which defines the depth of insertion into the handpiece 10.

Such fiber-optic light guide rods 22, and suitable materials and methods for their production, are known for example from DE 10 2013 208 838 B4 or DE 10 2004 034 603 B4 in the name of the Applicant, and may as shown therein be configured to be straight. They may, however, optionally also be configured to be angled at least in portions with respect to the longitudinal axis 22.4 of the light guide rod 22, particularly at their distal end, and also be configured to be additionally tapered at least in portions.

The above-described features describe typical dental curing units, which have already been employed for many years to cure tooth fillings.

According to the invention, the light guide unit 22 comprises at least one converter 24 which, in the operating state, converts the radiation 16.3 of the semiconductor lighting element 16.1 into a distal radiation 22.2 of the fiber-optic light guide rod 22 in such a way that the distal radiation 22.2 corresponds essentially to white or colored light, for example in the green, yellow or red spectral range. The converter 24 is operated in transmission, that is to say shone through, the blue light of the semiconductor lighting element 16.1 passing through the converter 24 and being converted at least partially into yellow light, for example when using a converter material consisting of a yellow-emitting Ce:YAG phosphor, so that in total white light or another colored light is emitted therefrom.

Modifications of the distal radiation 22.2, for example of the color appearance, that is to say the color location in the case of colored light or the color temperature in the case of white light, which occur for example due to ageing of the exciting light source, specifically the semiconductor lighting element 16.1, may be compensated for by adapting and/or readjusting the power of the semiconductor lighting element 16.1.

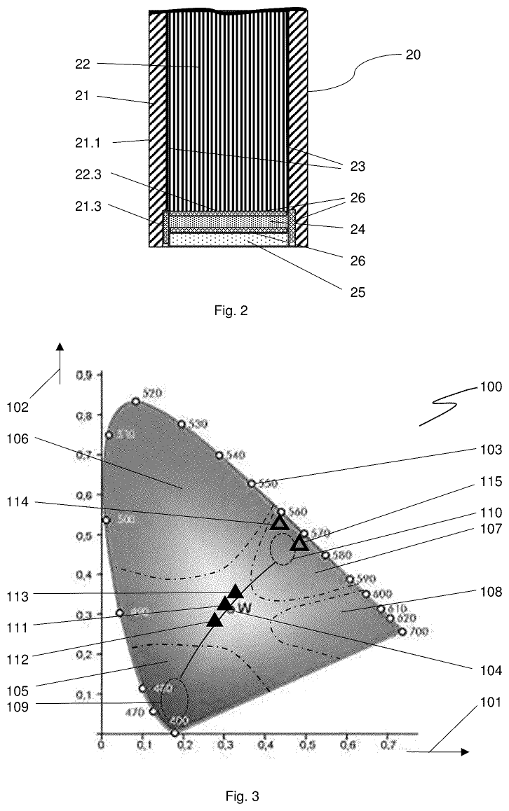

Further, in a preferred embodiment, the converter 24 is materially bonded directly onto the proximal end face 22.3 of the fiber-optic light guide rod 22. As already mentioned in the introduction, the converter 24 may be a platelet-like converter element, as may be seen by way of example in FIG. 1 and as a detail enlargement in FIG. 2, or it may however also be configured as a combination of different converter elements consisting of optionally different converter materials as the converter 24. In a further preferred variant, the converter ends flush with the sleeve 21 so that overhangs are avoided. The converter disc or discs are adhesively bonded by means of a second adhesive 26 to the proximal end face 22.3 of the fiber-optic light guide rod 22, or also to one another, without any bubbles. This second adhesive 26 ideally is clearly transparently curable and has self-levelling properties. In principle, UV-curing and/or thermally curing adhesive systems based on acrylate or epoxide are suitable. Furthermore, liquid silicones (liquid silicone rubber LSR) which crosslink under heat may also be envisaged. These are distinguished by good permanent elasticity and can also compensate for material differences in the thermal expansion coefficients between the fiber-optic light guide rod 22 and the converter 24.

Optionally, as shown by FIGS. 1 and 2, a cover 25, or disc, which fully covers the converter 24 may additionally also be adhesively bonded on the converter 24, this cover 25 ideally consisting of a chemically resistant glass (for example borosilicate glass), sapphire or quartz glass, and having a thickness in the range of from 30 μm to 500 μm, preferably from 50 μm to 200 μm, this cover 25 likewise being adhesively bonded to the converter 24 with the second adhesive 26.

As a further option, this cover 25 may have a reflection-reducing coating at least on the side facing towards the illumination unit 16. Such coatings are also known as N/4 layers, and are often applied as a layer sequence.

Ideally, the sleeve 21 has a widening of the inner diameter in the region of the converter 24 and/or of the cover 25, so that a defined adhesive reservoir 21.3 is formed, or formable. This simplifies the adhesive bonding process and helps to avoid waste due to excess adhesive on the converter 24, or on the cover 25.

The principle of the light conversion may be explained with a CIE color space diagram 100, as represented in FIG. 3.

FIG. 3 schematically shows in detail a CIE color space diagram 100 such as may be found in the technical literature, with the chromaticity axes cₓ 101 and c_{y} 102 and the white point W, or achromatic point 104, which is defined by the coordinates cₓ=0.33 and c_{y}=0.33. At the edge of the color space, the wavelength 103 is indicated in nm for visible light, starting with 400 nm in the blue range 105, through the green range 106 and the yellow range 107, to 700 nm in the red range 108.

If blue light is now emitted, typically between 400 nm and 500 nm, conventionally at 405 nm and/or 450 nm, which corresponds to the emission range 109 of the semiconductor lighting element 16.1, by at least partial conversion of blue light into yellow light, for example using an essentially yellow-emitting Ce-doped YAG phosphor, or a ceramic converter 24 consisting thereof, which corresponds to the converted emission 110, colored as well as white light may be generated along the connecting line between the emission range 109 and the converted emission 110.

By the nature and/or composition of the converter 24, the porosity of the converter 24, the percentage fraction of the dopants in the converter 24 and/or the thickness of the converter 24, various color locations between the blue excitation and emission in the yellow range, or color temperatures in the white range around the white point W, or achromatic point 104, may be generated. Thus, a more colored or warmer white light may be generated with more highly porous converter material or thicker material, and a cooler white 112 may be generated with thinner converter thicknesses or a lower porosity. A converter material which is less porous, or whose thickness is somewhat greater, accordingly gives a warm white 113. With a suitable balance between the blue excitation radiation passing through the converter 24 and the fraction of converted light, a neutral white 111 in the vicinity of the white point W, or achromatic point 104, may also be generated.

For conversion of blue light into white light, ceramic converter materials which contain Ce:YAG (yttrium aluminium garnet which is doped with cerium) or consist of this material may in particular be considered. In addition, it may also contain admixtures of gallium (Ga) in order to influence the emission, that is to say the wavelength or wavelength range of the secondary light emitted.

Depending on, or by the interplay of, the composition of the converter 24, its thickness, its porosity and/or proportion of phosphor, particularly in the case of composite materials as the converter 24, for example phosphor in glass or in silicone, colored light may for example also be achieved as distal radiation 22.2.

For example, a greenish overall emission 114 (see FIG. 3) may be achieved by Ce:LuAG, a lutetium aluminium garnet which is doped with cerium. On the other hand, a red-shifted yellow overall emission 115 (see FIG. 3) may be achieved with a converter 24 consisting of Ce:YAG, an yttrium aluminium garnet which is doped with cerium and contains admixtures of gallium (Ga). A green-shifted green overall emission may be achieved with a converter 24 consisting of Ce:LuAG, a lutetium aluminium garnet which is doped with cerium and contains admixtures of gadolinium (Gd).

The converter or converters 24, or converter elements, preferentially comprise or consist of a ceramic material and are described for example in the documents WO 13060731 A2 or WO 13139619 A1 in the name of the Applicant.

Exemplary embodiments (AB) of such ceramic converter materials are:

| Designation | Properties |
|---|---|
| AB1 | yellow-emitting converter 24 of the type Ce:YAG |
| AB2 | yellow-emitting converter 24 of the type Ce:YAG |
| | with increased Ce doping |
| AB3 | red-shifted yellow-emitting converter 24 of the type |
| | Ce:YAG with Ga doping |
| AB4 | green-shifted converter 24 of the type Ce:LuAG |
| AB5 | green-emitting converter 24 further shifted into the |
| | green range of the type Ce:LuAG with Gd doping |

The layer thickness of the converter 24 is typically from 80 μm to 150 μm, preferentially from 80 μm to 100 μm.

For the application described in the introduction, the following converter variants have been found to be advantageous, these preferentially having polished surfaces and an antireflection coating.

For instance, with a converter 24 according to the exemplary embodiments AB1 and AB2 above in an illumination system according to the invention with a thickness of about 80 μm, a white distal radiation may be obtained. With larger thicknesses, a distal radiation which becomes yellower with an increasing thickness may be obtained.

In order to obtain red-shifted or green-shifted distal radiations, converters 24 according to AB3 to AB5 with thicknesses of 80 μm may be used. Here again, the distal radiation becomes redder or greener, respectively, with an increasing thickness.

### REFERENCE NUMERALS

1 illumination system
10 handpiece
11 charging interface
12 charge monitoring unit
13 energy storage unit
14 illumination control unit
15 operating element
16 illumination unit
16.1 semiconductor lighting element
16.2 optical element
16.3 radiation
17 reception portion
17.1 fit
18 magnets
20 light guide unit
21 sleeve
21.1 fit
21.2 stop
21.3 adhesive reservoir
22 fiber-optic light guide rod
22.1 distal end face
22.2 distal radiation
22.3 proximal end face
22.4 longitudinal axis
23 first adhesive
24 converter
25 cover
26 second adhesive
100 CIE color space diagram
101 chromaticity axis cₓ
102 chromaticity axis c_{y}
103 wavelength
104 white/achromatic point W
105 blue range
106 green range
107 yellow range
108 red range
109 emission range
110 converted emission
111 neutral white
112 cool white
113 warm white
114 green overall emission
115 red/yellowish overall emission

What is claimed:

1. An Illumination system, comprising
a handpiece having an illumination unit and a semiconductor lighting element, wherein
the at least one semiconductor lighting element has in the operating state a radiation of essentially blue light in the wavelength range between 400 nm and 500 nm,
and a light guide unit which is releasably connected and/or connectable to the handpiece wherein
the light guide unit comprises a fiber-optic light guide rod, having a proximal end face and a distal end face, and a sleeve, wherein
the fiber-optic light guide rod is fixed in the sleeve by a first adhesive in a portion starting from the proximal end face (22.3), and wherein
the light guide unit comprises a one converter, the converter converting the radiation of the semiconductor lighting element in the operating state into a distal radiation of the fiber-optic light guide rod at its distal end face so that the distal radiation in the operating state has essentially white and/or color-neutral light, or colored light, of a one wavelength or of a one wavelength range in the visible spectral range.

2. The illumination system according to claim 1, wherein the converter is assigned to the proximal end face (22.3) of the fiber-optic light guide rod and at least partially overlaps the proximal end face (22.3), and is arranged facing towards the illumination unit when the light guide unit and the handpiece are connected.

3. The illumination system according to claim 1, further comprising of:
the converter comprises a ceramic converter material of the type Ce:YAG,
the converter comprises a ceramic converter material of the type Ce:YAG with additional Ga doping,
the converter comprises a ceramic converter material of the type Ce:LuAG,
the converter comprises a ceramic converter material of the type Ce:LuAG with additional Gd doping,
the converter comprises a ceramic converter material of the type Eu:SiN, Eu:SiON and/or Eu:SiAlON,
the converter comprises a converter material as a material composite of a glass matrix with particles of a phosphor incorporated therein,
the converter comprises a converter material as a material composite of a ceramic matrix with particles of a phosphor incorporated therein,
the converter comprises a converter material as a material composite of a polymer matrix with particles of a phosphor incorporated therein, or
the converter comprises a combination of at least two converter materials.

4. The illumination system according to claim 1, wherein the converter is formed as one or more disc-shaped converter elements consisting of a converter material, each of which has a thickness of from 30 μm to 200 μm.

5. The illumination system of claim 4, wherein the converter material, each of which has a thickness of from 50 μm to 150 μm.

6. The illumination system of claim 5, wherein the converter material, each of which has a thickness of from 80 μm to 100 μm.

7. The illumination system according to claim 4, wherein the one or more converter elements of the converter are adhesively bonded by a second adhesive onto the proximal end face of the fiber-optic light guide rod and/or to one another, the second adhesive being substantially optically clear, transparent and permanently elastic in the cured state.

8. The illumination system according to claim 1, further comprises on:
the color location in the case of colored distal radiation or the color temperature in the case of white or color-neutral distal radiation is adjustable by the composition of the converter and/or of the one or more converter elements,
the color location in the case of colored distal radiation or the color temperature in the case of white or color-neutral distal radiation is adjustable by the thickness of the converter and/or of the one or more converter elements, or
the color location in the case of colored distal radiation or the color temperature in the case of white or color-neutral distal radiation is adjustable by the porosity of the converter and/or of the one or more converter elements.

9. The illumination system according to claim 1, wherein a cover is applied on the converter or on the outer-lying converter element, in such a way that the converter, or the outer-lying converter element, is at least fully covered or overlapped, wherein
the cover comprises of a glass disc, a plastic disc, a sapphire disc or a quartz glass disc and has a thickness in the range of from 30 μm to 500 μm, and wherein this cover is likewise connected to the converter with the second adhesive.

10. The illumination system of claim 9, wherein the cover has a thickness in the range of from 50 μm to 200 μm.

11. The illumination system according to claim 9, wherein
the converter and/or the cover has a reflection-reducing coating at least on the side facing towards the illumination unit.

12. The illumination system according to claim 1, wherein a color filter is arranged between the converter and the proximal end face of the fiber-optic light guide rod.

13. The illumination system according to claim 1, wherein the sleeve has a widening of the inner diameter in the region of the converter and/or of the cover, so that an adhesive reservoir is formed or formable.
